(19) United States
(12) Patent Application Publication
(10) Pub. No.: US 2020/0367798 A1
(43) Pub. Date: Nov. 26, 2020

(54) WEARABLE DEVICE FOR DETERMINING PSYCHO-EMOTIONAL STATE OF USER DURING EVALUATION OR TESTING

(71) Applicant: Anthony Frolov, Bell Canyon, CA (US)

(72) Inventor: Anthony Frolov, Bell Canyon, CA (US)

(21) Appl. No.: 16/422,993

(22) Filed: May 25, 2019

Related U.S. Application Data
(63) Continuation-in-part of application No. 16/417,093, filed on May 20, 2019.

(51) Int. Cl. A61B 5/16; A61B 5/024; A61B 5/00; A61B 5/0476

(52) U.S. Cl. CPC A61B 5/165; A61B 5/02405; A61B 5/681; A61B 5/0205; A61B 5/168; A61B 5/167; A61B 5/0476

(57) ABSTRACT

Wearable devices and methods for determining a psycho-emotional state of a user are described herein. A wearable device for determining a psycho-emotional state of a user may include a plurality of sensors, an evaluation and testing module, and a processing module. The plurality of sensors may be configured to measure physiological parameters of the user. The evaluation and testing module may be configured to evaluate and test the user. The processing module may be configured to analyze the physiological parameters of the user during one of the evaluation or the testing. The processing module may be further configured to determine the psycho-emotional state of the user based on the analysis. The processing module may be configured to determine changes in the psycho-emotional state of the user. The processing module may be further configured to provide recommendations based on the changes in the psycho-emotional state of the user.

### CROSS-REFERENCE TO RELATED APPLICATION

This application is a Continuation-in-part of U.S. application Ser. No. 16/417,093 titled “METHODS FOR EVALUATING AND EDUCATING USERS” (Attorney Docket No. 1017.007US1), filed on May 20, 2019. The aforementioned application is incorporated herein by reference in its entirety for all purposes.

### TECHNICAL FIELD

The present disclosure relates generally to determining and correcting a psycho-emotional state of users and specifically to methods and wearable devices to determine and control a psycho-emotional state of users.

### BACKGROUND

Conventional wearable devices, such as fitness trackers, are widely used by users to track physical parameters, such as a pulse, a blood oxygen level, distance walked, steps, and so forth. The fitness trackers usually provide the measurement results in the form of diagrams showing change of each measured parameter in time. However, while the fitness trackers work well for determining physical parameters of users, they cannot determine a psycho-emotional state of the users or identify likelihood of a medical disorder in the users.

People with various mental conditions may have specific behavioral patterns or disorders of a motor function and often respond to changes in their psycho-emotional state in a way that is not easily detectable by others. For example, the behavior of people with an autism spectrum disorder or mental retardation differs significantly from the behavior of healthy people. It is difficult for an observer to determine how the psycho-emotional state of the people having the disorder changes. It is also difficult to understand the changes in the psycho-emotional state of a person with tonic regulation disorders, such as cerebral palsy, or of an elderly person.

In some cases, parents may face difficulties in determining whether a child has any signs of a mental disorder. Conventionally, the child needs to visit a doctor who tests the child to determine whether the child has a mental disorder. Currently, there are no practical solutions for determining a psycho-emotional state of a person by based on physiological parameters collected by sensors of a wearable device.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Provided are wearable devices, methods, and systems for determining a psycho-emotional state of a user. In some example embodiments, a wearable device for determining a psycho-emotional state of a user may include a plurality of sensors, an evaluation and testing module, and a processing module. The plurality of sensors may be configured to measure physiological parameters of the user. The evaluation and testing module may be configured to evaluate and test the user. The processing module may be configured to analyze the physiological parameters of the user during one of the evaluation or the testing. The processing module may be further configured to determine the psycho-emotional state of the user based on the analysis. The processing module may be configured to determine changes in the psycho-emotional state of the user based on the psycho-emotional state. The processing module may be further configured to provide recommendations based on the changes in the psycho-emotional state of the user.

In some example embodiments, a method for determining a psycho-emotional state of a user during evaluation may commence with providing a plurality of sensors configured to measure physiological parameters of the user during the evaluation. The method may further include evaluating and testing the user. The method may further include analyzing the physiological parameters of the user during one of the evaluation or the testing. The method may continue with determining the psycho-emotional state of the user based on the analysis. The method may further include determining changes in the psycho-emotional state of the user based on the psycho-emotional state. The method may continue with providing recommendations based on the changes in the psycho-emotional state of the user.

In some example embodiments, a non-transitory computer readable storage medium having embodied thereon a program executable by a processor to perform a method for determining a psycho-emotional state of a user during evaluation is provided. The method may commence with may commence with providing a plurality of sensors configured to measure physiological parameters of the user during the evaluation. The method may further include evaluating and testing the user. The method may further include analyzing the physiological parameters of the user during one of the evaluation or the testing. The method may continue with determining the psycho-emotional state of the user based on the analysis. The method may further include determining changes in the psycho-emotional state of the user based on the psycho-emotional state. The method may continue with providing recommendations based on the changes in the psycho-emotional state of the user.

Additional objects, advantages, and novel features will be set forth in part in the detailed description section of this disclosure, which follows, and in part will become apparent to those skilled in the art upon examination of this specification and the accompanying drawings or may be learned by production or operation of the example embodiments. The objects and advantages of the concepts may be realized and attained by means of the methodologies, instrumentalities, and combinations particularly pointed out in the appended claims.

### BRIEF DESCRIPTION OF THE DRAWINGS

Embodiments are illustrated by way of example and not limitation in the figures of the accompanying drawings, in which like references indicate similar elements and in which:

FIG. 1 illustrates an environment within which wearable devices, system, and methods for determining a psycho-emotional state of a user can be implemented, in accordance with some embodiments.

FIG. 2 is a block diagram showing various modules of a wearable device for determining a psycho-emotional state of a user, in accordance with certain embodiments.

FIG. 3 is a schematic diagram a wearable device for determining a psycho-emotional state of a user, in accordance with certain embodiments.

FIG. 4 is a flow chart illustrating a method for determining a psycho-emotional state of a user, in accordance with an example embodiment.

FIG. 5 is a schematic diagram illustrating a method for determining a psycho-emotional state of a user to develop an association between a visual representation of objects and real objects, according to an example embodiment.

FIG. 6 is a schematic diagram illustrating a method for determining a psycho-emotional state of a user to develop an association between a visual representation of actions and real actions, according to an example embodiment.

FIG. 7 is a schematic diagram illustrating a method for determining a psycho-emotional state of a user to develop an association between an audible representation of objects or actions and real objects or actions, according to an example embodiment

FIG. 8 is a schematic diagram illustrating a method for determining a psycho-emotional state of a user to develop a non-stereotypical behavior with regard to objects shown on a screen of a computing device, according to an example embodiment.

FIG. 9 shows a computing system that can be used to implement a method for determining a psycho-emotional state of a user, according to an example embodiment.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical, and electrical changes can be made without departing from the scope of what is claimed. The following detailed description is, therefore, not to be taken in a limiting sense, and the scope is defined by the appended claims and their equivalents.

The present disclosure provides wearable devices, methods, and systems for determining a psycho-emotional state of a user. These wearable devices, methods and systems may be used by parents and teachers when conducting educatory, corrective, and development trainings for children and adults having autistic disorders, logopedic disorders, mental disorders, age-related disorders, and the like. The wearable devices, methods and systems may be applied to determine and control a psycho-emotional state of users to teach and socially adapt the users and develop required skills of the users based on the determined psycho-emotional state.

A wearable device for determining a psycho-emotional state of a user (hereinafter referred to as a wearable device) may include any wearable device configured to be worn on/attached to/contact with a body of the user. Example wearable devices for determining a psycho-emotional state of a user may include a bracelet, a heart monitor, a smartwatch, a smartphone, and so forth. The wearable device may include one or more sensors for measuring physiological parameters of the user. The sensors may be attached to or build into a housing of the wearable device. In another example embodiment, the sensors may be not placed into the housing and may be configured to be directly attached to or contact with the body of the user. The physiological parameters may include a heart rate (pulse frequency), a blood pressure, an electrical conductivity of a skin, a blood oxygen level, sleep time, sleep quality, a blood sugar level, and so forth.

The wearable device may further have an evaluation and testing module and a processing module. The evaluation and testing module may receive the physiological parameters measured by the sensors and provide the physiological parameters to the processing module. The processing module may analyze the physiological parameters of the user and determine the psycho-emotional state of the user based on the analysis. For example, a detailed pulsogram, i.e., a diagram of the pulse frequency, of the user showing change of the pulse frequency over time may be built by the processing module based on the measured heart rate. Furthermore, the processing module may generate diagrams for any other measured physiological parameter of the user. The analysis may be performed by analyzing the change of the physical parameters shown by the pulsogram and/or other diagrams. The psycho-emotional state may include apathy, critical excitation, aggression, psychological despondency, and so forth. The information on the psycho-emotional state of the user may be provided for review to an inspecting person, such as a parent, a teacher, a physiologist, and so forth. Specifically, the information on the psycho-emotional state may be provided to a user device of the inspecting person, such as a smartphone, a personal computer (PC), a tablet PC, and so forth. The inspecting person may be located in proximity to the user, e.g., may teach or train the user. In this case, as the wearable device continuously tracks the psycho-emotional state of the user, the information on the psycho-emotional state may be provided to the user device of the inspecting person in real time. The inspecting person may review the information on the psycho-emotional state of the user and adapt a teaching or training session for the user in real time.

Therefore, the wearable device may allow professionals or inspecting persons who directly interact with people having various mental disorders to significantly increase the effectiveness of professional help and monitor critical changes in the psycho-emotional state of the people. For example, teachers of inclusive and correctional classes may use the wearable devices, methods and systems of the present disclosure to track changes in the psycho-emotional state of users having various disorders (for example, autism). For example, the wearable device may help track the attention concentration level of children and prevent emotional breakdowns as the inspecting person may be able to detect the emotional breakdowns at the very beginning. Health professionals and support services may use the wearable devices, methods, and systems of the present disclosure to recognize changes in the psycho-emotional state of elderly patients and other patients who do not respond to stimuli in a way conventional for healthy people, thus avoiding critical situations.

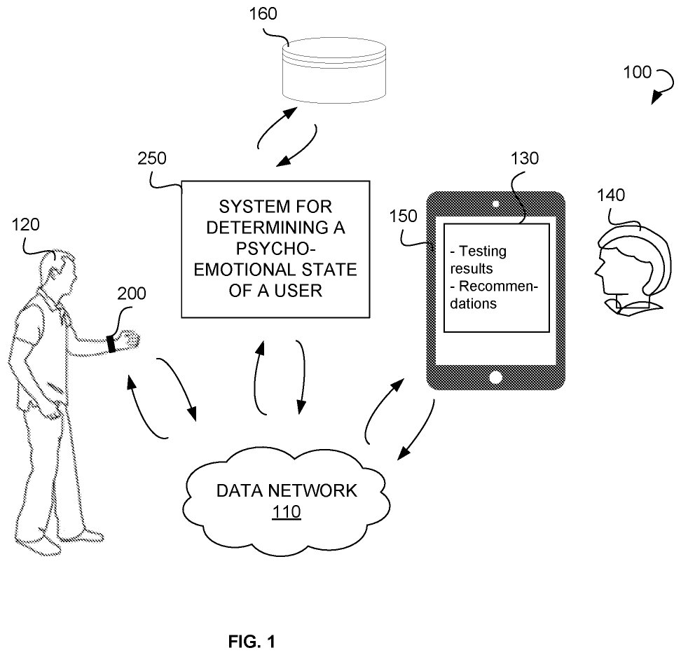

Referring now to the drawings, FIG. 1 illustrates an environment 100 within which wearable devices, methods, and systems for determining a psycho-emotional state of a user can be implemented. The environment 100 may include a data network 110, a user 120, a wearable device 200 associated with the user 120, a system for determining a psycho-emotional state of a user shown as a system 250, a database 160 associated with the system 250, an inspecting person 140, and a user device 150 associated with the inspecting person 140. The wearable device 200 may include a bracelet, a smartwatch, a fitness tracker, and so forth. The user device 150 may include a smartphone, a PC, a tablet PC, a personal wearable device, a computing device, and so forth.

The data network 110 may include the Internet, a computing cloud, and any other network capable of communicating data between devices. Suitable networks may include or interface with any one or more of, for instance, a local intranet, a Personal Area Network, a Local Area Network, a Wide Area Network, a Metropolitan Area Network, a virtual private network, a storage area network, a frame relay connection, an Advanced Intelligent Network connection, a synchronous optical network connection, a digital T1, T3, E1 or E3 line, Digital Data Service connection, Digital Subscriber Line connection, an Ethernet connection, an Integrated Services Digital Network line, a dial-up port such as a V.90, V.34 or V.34bis analog modem connection, a cable modem, an Asynchronous Transfer Mode connection, or a Fiber Distributed Data Interface or Copper Distributed Data Interface connection. Furthermore, communications may also include links to any of a variety of wireless networks, including Wireless Application Protocol, General Packet Radio Service, Global System for Mobile Communication, Code Division Multiple Access or Time Division Multiple Access, cellular phone networks, Global Positioning System, cellular digital packet data, Research in Motion, Limited duplex paging network, Bluetooth radio, or an IEEE 802.11-based radio frequency network. The data network can further include or interface with any one or more of Recommended Standard 232 (RS-232) serial connection, an IEEE-1394 (FireWire) connection, a Fiber Channel connection, an IrDA (infrared) port, a Small Computer Systems Interface connection, a Universal Serial Bus connection or other wired or wireless, digital or analog interface or connection, mesh or Digi® networking. The data network may include a network of data processing nodes, also referred to as network nodes, that are interconnected for the purpose of data communication.

The wearable device 200 may contact a body of the user 120. The wearable device 200 may have a plurality of sensors and may measure physiological parameters of the user 120. The physiological parameters of the user 120 may be analyzed to determine the psycho-emotional state of the user 120. The user 120 may be further evaluated and tested to establish initial skills of the user with respect to an object or an action. The evaluation and testing of the user 120 may include testing the user 120 to determine whether the user 120 understands meaning of various objects and actions and is able to associate the objects and actions with similar objects and actions. Based on the psycho-emotional state of the user 120, recommendations 130 associated with the psycho-emotional state of the user 120 may be provided to the inspecting party 140. Specifically, the recommendations 130 may be presented on a display of the user device 150 of the inspecting party 140. The recommendations 130 may include testing results, analysis results, information on correction of the psycho-emotional state of the user 120, recommendations on correction of the psycho-emotional state of the user 120, and so forth.

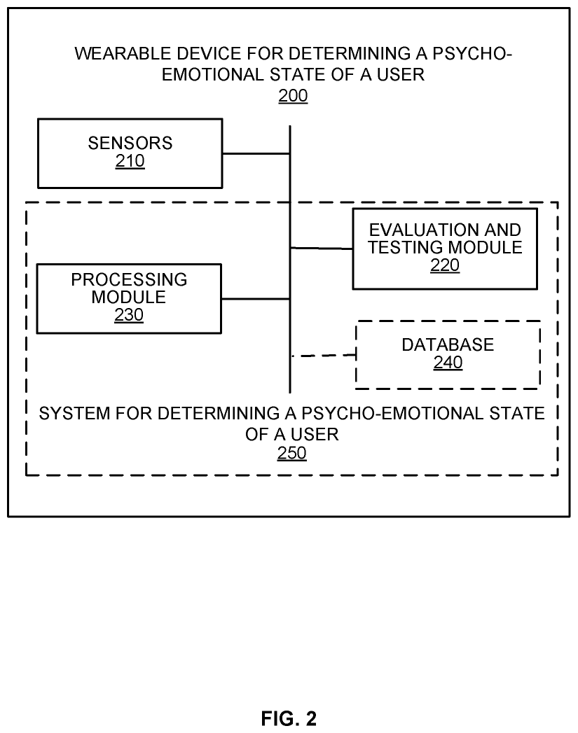

FIG. 2 is a block diagram showing various modules of a wearable device 200 for determining a psycho-emotional state of a user, in accordance with certain embodiments. In an example embodiment, the wearable device may be provided in form of a bracelet. Specifically, the wearable device 200 may include one or more sensors 210, an evaluation and testing module 220, a processing module 230, and optionally a storage unit shown as a database 240. In an example embodiment, each of the evaluation module 210 and the processing module 230 may include a programmable processor, such as a microcontroller, a central processing unit, and so forth. In example embodiments, each of the evaluation module 220 and the processing module 230 may include an application-specific integrated circuit or programmable logic array designed to implement the functions performed by the wearable device 200.

In an example embodiment, the wearable device 200 may include a system 250 for determining a psycho-emotional state of a user. In this embodiment, the evaluation and testing module 220, the processing module 230, and the database 240 may be components of the system 250. The system 250 may be configured in the form of an application running on the wearable device 200. In some embodiment, the system 250 may reside in a computing cloud and may communicate with the wearable device 200 via an application programming interface (API).

The wearable device 200 may be configured to be worn by a user. The user may include a person diagnosed with a medical condition. The medical condition may include one of a chronic disease, a mental condition, an autism spectrum disorder, and so forth. The sensors 210 of the wearable device 200 may be configured to measure physiological parameters of the user. The physiological parameters may include one or more of a pulse, a blood pressure, a blood oxygen level, an electrical conductivity of skin, and so forth.

The evaluation and testing module 220 may be configured to evaluate and test the user. The processing module 230 may be configured to analyze the physiological parameters of the user during one of the evaluation or the testing. Based on the analysis, the processing module 230 may determine the psycho-emotional state of the user. More specifically, the processing module 230 may analyze the physiological parameters of the user using a predetermined algorithm. The processing module 230 may have access to the database 240 in which historical data associated with the user and/or further users may be stored. The predetermined algorithm may be created based on the historical data and may include information on correlation between a psycho-emotional state and/or a mental or physical disorder of a person and each of physiological parameters separately or a combination of the physiological parameters. The processing module 230 may match the physiological parameters of the user or the combination of the physiological parameters of the user with physiological parameters indicative of a particular psycho-emotional state and/or mental or physical disorder. Based on the correlation and matching, the processing module 230 may determine the psycho-emotional state of the user.

In an example embodiment, the processing module 230 may perform the analysis of the physiological parameters of the user using Artificial Intelligence (AI) and machine learning. An AI module and the machine learning module may be in communication with the processing module 230. The AI module and the machine learning module may be configured to be trained on historical data. Specifically, the AI module and the machine learning module may be configured to be trained on historical data associated with physiological parameters using extrapolation of historical data to the physiological parameters measured for the user, a data anomaly detection technique, predictive analytics, a Bayes algorithm, a graph theory, a pattern recognition technique, and any other methods applicable in AI and machine learning.

Based on the psycho-emotional state, the processing module 230 may determine changes in the psycho-emotional state of the user. The changes may be determined based on comparison of the physiological parameters of the user with historical physiological parameters of the user. The historical physiological parameters of the user may include physiological parameters of the user preliminarily collected by the wearable device 200 and stored in the database 240. In an example embodiment, the wearable device 200 may continuously collect the physiological parameters of the user and store the collected physiological parameters to the database 240 as user data. In an example embodiment, the processing module 230 may further analyze historical physiological parameters associated with further users. The further users may include healthy people and people having mental disorders.

The processing module 230 may be configured to provide recommendations based on the changes in the psycho-emotional state of the user. In an example embodiment, the processing module 230 may visualize the changes on a user interface of a user device. The user device may be associated with an inspecting person who performs evaluation, testing, training, or teaching of the user. The psycho-emotional state of the user may be indicative of one of a physical exertion, an emotional exertion, a level of concentration, an emotional state, a sensory overload, an excitement, an apathy, and so forth. Therefore, the recommendations may include information on the current psycho-emotional state of the user and actions to be taken by the inspecting person to correct the psycho-emotional state or prevent the development of one or more of critical excitement, apathy, and so forth.

In an example embodiment, the evaluation and testing of the user performed by the evaluation and testing module 220 may include performing an initial evaluation of the user to establish initial skills with respect to an object or an action. The initial evaluation may include identifying a disorder of the user. The disorder may include at least one of the following: an autism spectrum disorder, a speech disorder, a mental disorder, a logopedic disorder, an age-related disorders, and the like.

In particular, the user, such as a child or an adult, may be initially evaluated to establish initial skills of the user with respect to an object or an action. The user may be tested to determine the level of knowledge and the level of retention of material of the user. Users having mental disorders may face difficulties in developing a correct association between a word denoting an action and the action itself or between a word denoting an object and the object itself. The evaluation of the user may include testing the user to determine whether the user understands the meaning of various objects and actions and is able to associate the objects and actions with similar objects and actions. The testing may include providing predetermined questions on a predetermined topic and receiving responses to the questions from the user. In an example embodiment, the evaluation of the user may be performed to determine whether the user likely has an autism spectrum disorder, a mental disorder, or the like, as well as the severity of the disorder. Furthermore, the results of evaluation may be provided to a teacher or a parent in accordance with a predetermined scale related to the autism spectrum disorders. The scale may be ranged from the state of a non-contact person to the state of a seemingly normal person having some autism-associated problems. The results of evaluation may show the likelihood of presence/absence of the disorder and the heaviness of the disorder.

Based on results of the evaluation, the evaluation and testing module 220 may select a skill development plan for the user designed to improve the initial skills with respect to the object or the action. The evaluation and testing module 220 may provide, to the user, an augmented reality (AR) application for an AR-enabled user device, such as a smartphone or a tablet PC. Specifically, the user may have or may be provided with (e.g., by a teacher) the AR-enabled user device on which the AR application may be run. The evaluation and testing module 220 may be further configured to provide the object or the action to the user according to the skill development plan. In particular, the skill development plan may include a training program and a set of cards, stickers, or other items. The cards of the set may have images representing an object or an action. Each of the cards may have a tag associated with the AR application. The tag may include an identifier placed on the cards.

The evaluation and testing module 220 may determine that the user is perceiving the object or the action via the AR-enabled user device. The object or the action may be associated with a card or a sticker and may have an identifier. In particular, the user may direct a camera of the AR-enabled user device on the card. The camera may detect the identifier on the card. The evaluation and testing module 220 may ascertain the identifier associated with the object or the action by the AR application. Based on the identifier, the evaluation and testing module 220 may activate, via the AR application, an interactive session designed to improve the initial skills with respect to the object or the action.

The interactive session may include providing interactive animation materials, e.g., in the form of an AR three-dimensional virtual game, to the user to improve the initial skills of the user with respect to the object or the action. The interactive animation materials may include a picture/animation of the object or the action and an explanation of the meaning, specific features, or purpose of the object or the action. The picture/animation of the object or the action may be displayed multiple times to the user to strengthen rote memorization of meaning of the object or the action. Each time the object or the action is displayed to the user, the appearance, such as color, size, and type of the object or the action, and location of the object or the action on a screen of the AR-enabled device may be changed. The change of the appearance and location of the object or the action may help the user to develop the ability to associate the appearance of the object or the action shown on the picture with other similar objects or actions of the same type.

More specifically, in an example embodiment, the interactive session may include presenting a word and displaying a plurality of further objects or actions related to the word. In a further example embodiment, the interactive session may include repeated pronunciations of a word by varying voices having different voice parameters. The voice parameters may include a speed, a vocal range, and so forth. In an example embodiment, the interactive session may include presenting buttons having varying parameters. The parameters may include a shape, a color, a size, a font, a location on a screen of the AR-enabled user device, and so forth. In a further example embodiment, the interactive session may include presenting, to the user via the AR-enabled user device, an animation designed to explain a meaning of the object or the action. In an example embodiment, the interactive session may include providing a test to the user, receiving a response to the test from the user, and, based on the response, selectively providing additional objects, actions, or tests to reinforce the response of the user.

The evaluation and testing module 220 may further perform a follow-up evaluation of the user to establish an improvement in the at least one of the initial skills. Specifically, during the interactive session, a follow-up evaluation of the user may be performed to establish an improvement in the initial skills. The follow-up evaluation may show objects or actions that were unsuccessfully learned by the user. These objects or actions may be repeatedly displayed to the user during the current or following interactive session. Furthermore, other tasks or questions may be selected for the user to teach the unsuccessfully learned material (objects or actions) to the user.

The wearable device 200 may be used by an inspecting person (a teacher or a parent) to identify and control the psycho-emotional state of children having autism spectrum disorders and other mental disorders. A child with autism has a specific system of reactions to external stimuli and changes in surrounding conditions, which are significantly different from reactions inherent in a child having no autism. It is difficult for the inspecting person to determine how much the child with autism is concentrated, attentive, and involved into the studying process, or determine whether the child is currently in a destructive emotional state, such as a sensory overload, physical exertion, or emotional exertion. The wearable device 200 and the system 250 associated with the wearable device 200 may help the inspecting person to determine and control the psycho-emotional state of children and adults who have psychoneurology disorders, including tonic regulation disorders. The analysis performed based on the physiological parameters of the user may help optimize the teaching process due to obtaining by the inspecting person reliable information on the psycho-emotional state of users during the teaching or training process. More specifically, the inspecting person may react to changes of the psycho-emotional state of users in real time and adapt/modify the teaching or training process based on the current psycho-emotional state of the users.

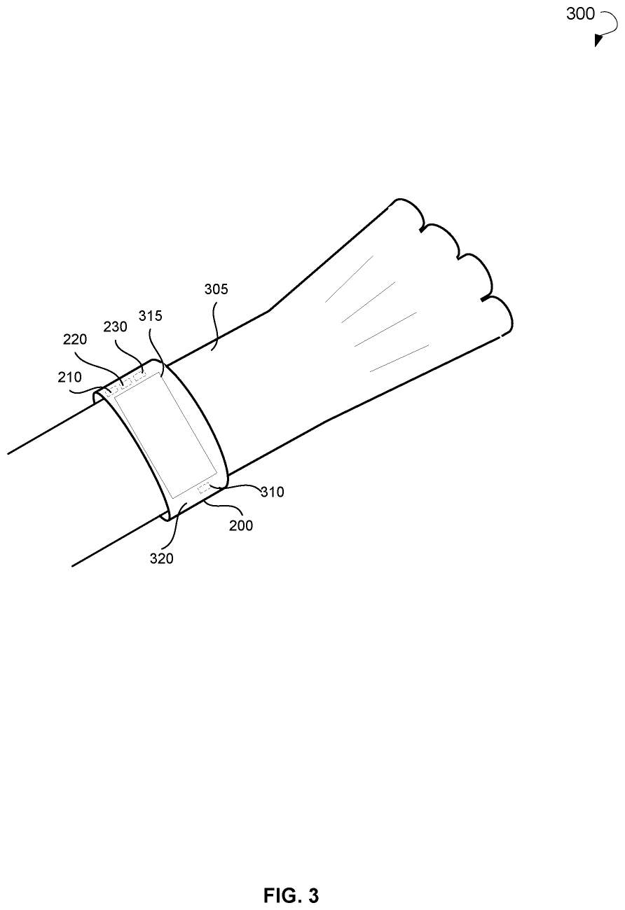

FIG. 3 is a schematic diagram 300 illustrating a wearable device 200 for determining a psycho-emotional state of a user, according to an example embodiment. The wearable device 200 may be provided in the form of a bracelet configured to be worn on a wrist 305 of a user. The wearable device 200 may have a housing 320 configured to enclose all elements of the wearable device 200. The wearable device 200 may have one or more sensors 210, an evaluation and testing module 220, and a processing module 230. The sensors 210 may include a heart-rate sensor, a blood pressure sensor, a blood oxygen sensor, a skin conductivity sensor, a blood glucose meter, a temperature sensor, a wearable chemical/electrochemical sensor for real-time non-invasive monitoring of electrolytes and metabolites in sweat, tears, or saliva, and the like. In an example embodiment, the sensors 210 may include non-invasive sensors or invasive sensors.

In an example embodiment, the wearable device 200 may further have a power source 315 and optionally a display 315. The display 315 may be configured to display information on physiological parameters of the user measured by the sensors 210. In some embodiment, the processing module 230 may provide notifications to the user on the display 315. In an example embodiment, notifications on the display 315 may be provided to draw the attention of an inspecting person and inform about changes in the psycho-emotional state of the user.

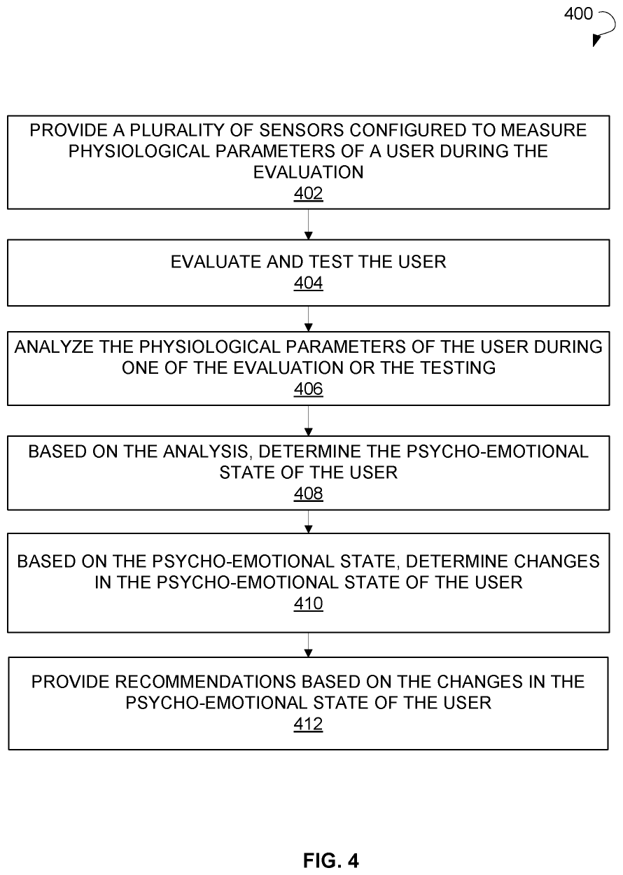

FIG. 4 is a flow chart illustrating a method 400 for determining a psycho-emotional state of a user during evaluation, in accordance with certain embodiments. The user may include a person diagnosed with a medical condition. The medical condition may include one of a chronic disease, a mental condition, an autism spectrum disorder, and so forth. In some embodiments, the operations may be combined, performed in parallel, or performed in a different order. The method 400 may also include additional or fewer operations than those illustrated. The method 400 may be performed by processing logic that may comprise hardware (e.g., decision making logic, dedicated logic, programmable logic, and microcode), software (such as software run on a general-purpose computer system or a dedicated machine), or a combination of both.

The method 400 may commence with providing a plurality of sensors at operation 402. In an example embodiment, the wearable device may include a bracelet. The plurality of sensors may be disposed in a housing of the wearable device. The sensors may be configured to measure physiological parameters of the user during the evaluation.

The method 400 may further include evaluating and testing the user at operation 404. The method 400 may further include analyzing the physiological parameters of the user during one of the evaluation or the testing at operation 406. The method 400 may continue with determining the psycho-emotional state of the user based on the analysis at operation 408. The method 400 may further include determining changes in the psycho-emotional state of the user based on the psycho-emotional state at operation 410.

The method 400 may further include providing recommendations based on the changes in the psycho-emotional state of the user at operation 412. The recommendations may be selected for the user based on the psycho-emotional state. In an example embodiment, the recommendation may be provided to an inspecting person who teaches or trains the user.

In an example embodiment, evaluating and testing of the user may include performing an initial evaluation of the user to establish initial skills with respect to an object or an action. The initial evaluation may be performed by an evaluation and testing module. In an example embodiment, the initial evaluation may include identifying a disorder of the user. The disorder may include at least one of the following: an autism spectrum disorder, a speech disorder, a mental disorder, and so forth. In an example embodiment, the initial evaluation of the user may be directed to detecting a presence of a mental condition, determining a mental condition, determining a physiological condition of the user, and determining a knowledge of the user with respect to objects and actions.

The method 400 may continue with selecting a skill development plan for the user. The skill development plan may be designed to improve the initial skills with respect to the object or the action. The skill development plan may be selected based on results of the evaluation of the user by a person that educates the user, such as a parent or a teacher. In an example embodiment, the skill development plan may be selected for the user by a processing module based on the results of the initial evaluation.

Specifically, a plurality of skill development plans may be preliminarily developed for a plurality of possible disorders. In this case, the skill development plan may be selected from the preliminarily developed skill development plans based on the results of the initial evaluation or the psycho-emotional state of the user. In another embodiment, the skill development plan may be developed specifically for the user based on the initial level of skills and knowledge, disorders of the user, and/or the psycho-emotional state of the user.

In an example embodiment, the skill development plan may include a set of items, such as cards, stickers, plates, sheets, articles, and the like. An AR tag may be placed onto each item of the set. The AR tag may be an identifier configured to be read by a camera of an AR-enabled user device. The skill development plan may further include a training program, an instruction on use of the set of items, such as an order of providing the items to the user, a list of tasks, a list of lessons and a list of specific items to be provided to the user at each lesson, a number of days of a training, and so forth. The skill development plan may include a description of topics, objects, and actions to be studied by the user, a description of the sequence of objects and actions to be presented to the user, a periodicity of presenting objects and actions to the user, and so forth.

An image may be placed on each of the items. The image (a photo, a picture, or other visual image) may depict the object or the action. The object may include any article the meaning of which the user needs to learn (e.g., a flower, a wardrobe, a car, an animal, a book, and so forth). The action may include any action the meaning of which the user needs to learn (e.g., running, walking, swimming, flying, doing, and so forth). The action may be depicted by showing a character performing the action (e.g., a running cartoon character may be shown for the action “run”).

The method 400 may further include providing, to the user, an AR application for the AR-enabled user device. The AR-enabled user device may include a smartphone or a tablet PC.

The method 400 may continue with providing the object or the action to the user according to the skill development plan. The object or the action may be associated with a card, a sticker, a sample representation, a sound, and so forth. Specifically, the object or the action may be depicted on the card, the sticker, or the sample representation, or the object or the action may be presented via a sound. The card, the sticker, or the sample representation may be placed by the user in front of the camera of the AR-enabled user device. The camera of the AR-enabled user device may detect the identifier in the card, the sticker, or the sample representation.

The method 400 may further include determining that the user is perceiving the object or the action via the AR-enabled user device. The object or the action may be associated with the identifier. Specifically, based on capturing of the identifier, the AR-enabled user device may determine that the user is perceiving the object or the action via the AR-enabled user device. The identifier associated with the object or the action may be ascertained by the AR application. Based on the identifier, an interactive session may be activated via the AR application. The interactive session may be designed to improve the initial skills with respect to the object or the action.

The user may interact with the AR application via an interactive interface during the interactive session. Specifically, the user may select answers which the user believes are correct, respond to questions, solve tasks and play quest games related to the object or action, and so forth.

In a further example embodiment, the interactive session may include providing a test, a question, and/or a task to the user, receiving a response from the user, and selectively providing, based on the response, additional objects, actions, or tests to reinforce the response of the user. The tests, questions, and/or tasks may include or be associated with explanations or specifications relating to the object or action and may further describe the meaning and other peculiarities of the object or action.

In an example embodiment, the interactive session may be performed by presenting a word to the user. The word may be associated with the object or the action. The word may be presented via the AR-enabled user device by displaying the word on a screen or reproducing the word using an audio unit of the AR-enabled user device. The word presented to the user may be accompanied by displaying a plurality of further objects or actions related to the word. In addition to presenting the word, the explanation of the word, i.e., the explanation of the object or action denoted by the word, the purpose of the object or action, and other features/properties of the objects or actions may be presented to the user via the AR-enabled user device. The AR application may present the object or action and accompanying explanations in a form of interactive animation. The interactive animation may be provided in the form of AR objects on a screen of the AR-enabled user device. The interactive animation may be a combination of visual and audio information directed to stimulate visual and auditory channels of information perception of the user. Furthermore, the motor memory of the user may be engaged when the user needs to manipulate the user device or provide responses in the AR application. The activation of multiple channels of information perception and the motor memory of the user may increase the efficiency of perception of information and may help the user to learn the material.

In a further example embodiment, the interactive session may include repeated pronunciations of a word by varying voices having different voice parameters. The voice parameters may include a speed, a vocal range, and so forth.

In a further example embodiment, the interactive session may include presenting buttons having varying parameters. The varying parameters may include a shape, a color, a size, a font, and a location on a screen of the AR-enabled user device.

In an example embodiment, the interactive session may include presenting, to the user via the AR-enabled user device, an animation designed to explain a meaning of the object or the action.

The method 400 may continue with performing a follow-up evaluation of the user. The follow-up evaluation of the user may be performed to establish an improvement in at least one of the initial skills of the user. For example, the follow-up evaluation may include determining whether the user understands the meaning of the object or action. Based on the follow-up evaluation, the skill development plan may be modified. Specifically, it may be determined that some of the objects and actions are not learned by the user. These objects and actions may be placed into the skill development plan for further learning by the user. Furthermore, additional objects, actions, tasks, questions, tests, and game quests may be added into the skill development plan. Therefore, the process of teaching of the user may include the combination of training (in the form of presenting objects and actions to the user) and testing (in the form of evaluating whether the user provided correct responses to questions) of the user. The testing may be performed throughout the training, namely, at the beginning of the training/lesson to test an initial level of knowledge of the user and a mental state, after explanation of objects and actions to determine the level of retention of the material related to the objects and actions, and at the end of the training/lesson to determine the results of the training/lesson.

In an example embodiment, the skill development plan may be dynamically adapted based on the follow-up evaluation. Specifically, objects and actions that were not learned by the user as shown by the follow-up evaluation may be added to further interactive sessions of the skill development plan, and further objects and actions to be learned by the user may be added to the skill development plan based on the follow-up evaluation. The actions made by the user in the AR application, mistakes of the user, and correct answers may be recorded to the database as user data. The results of the follow-up evaluation may be further added to the user data in the database. The dynamic modification of the skill development plan may be performed based on the user data continuously collected by one or more of the AR application, the AR-enabled user device, a teacher, a parent, and so forth.

Additionally, the AR application may provide notifications to the teacher or parent informing the results of the follow-up evaluation and recommending further objects, actions, lessons, and topics to the learned by the user.

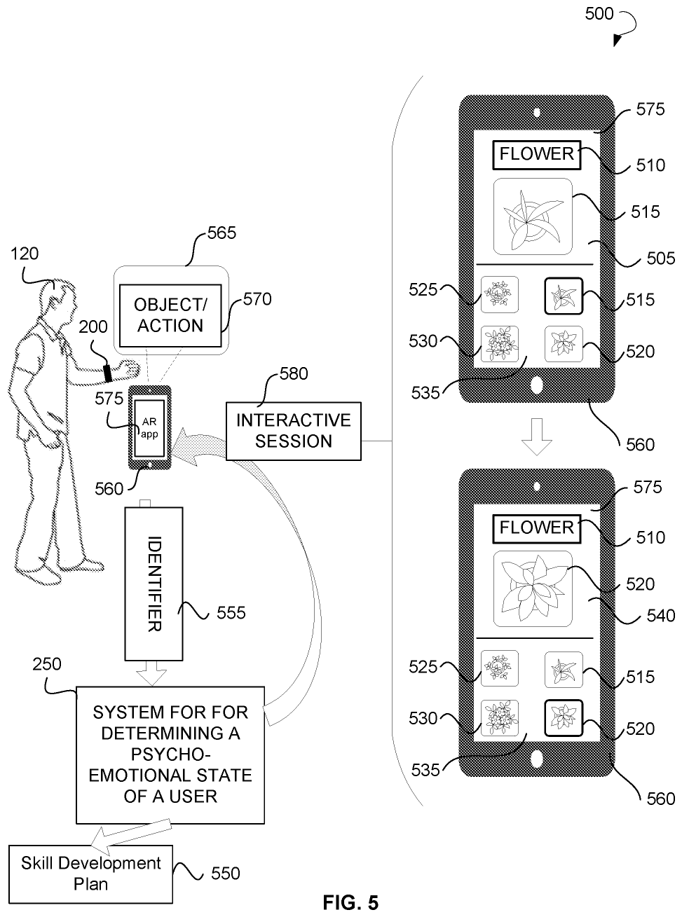

FIG. 5 is a schematic diagram 500 illustrating a method for determining a psycho-emotional state of a user to develop an association between a visual representation of objects and real objects, i.e., the objects per se, according to an example embodiment. In an example embodiment, the method may be used for educating users suffering from autism spectrum disorders and other mental disorders to identify objects using mobile applications, computer programs, and Internet services. The method solves the problem of inability of users to develop an association between real objects and visual images of the objects.

The users having aforementioned disorders may be able to create a limited association of real objects with visual images of real objects or may be unable to create any association. For example, if the picture shows a red cabinet, the user may consider only a cabinet of the same shape and the same red color to be the cabinet. The user may be unable to recognize a cabinet of other shape, size, or color to be a cabinet. Such peculiarities of perception by the user may limit the ability of the user to understand the real world and use services for training the users, such as mobile applications, computer programs, and Internet services.

To expand the associative array for objects of the user, the method includes demonstrating an object multiple times. Each time the object is presented, the shape, size, and color of the object may be changed. For example, when the object “flower” is described, multiple images of a flower may be sequentially presented to the user, and each flower on the images may have a differing shape, size, and color.

As shown on FIG. 5, a user 120 may wear a wearable device 200. The wearable device 200 may measure physiological parameters of the user 120. The system 250 may analyze the physiological parameters, determine the psycho-emotional state of the user, and select a skill development plan 550 for the user based on the psycho-emotional state.

The skill development plan 550 may include a set of items, such as cards, stickers, or articles. Each of the items may depict an object or an action and may include an AR tag. The AR tag may be an identifier 555 placed on each of the items. Each of the items may have its individual identifier. The user 120 may activate a camera of the user device 560 and direct the camera view to an item 565 (e.g., a card) depicting an object/action 570. The user device 560 may capture the identifier 555 of the item 565 and activate the AR application 575 based on the identifier 555. The AR application 575 may initiate an interactive session 580 associated with the identifier 555 and designed to improve the initial skills with respect to the object/action 570.

The user 120 may interact with an AR application 575 running on a user device 560. A user interface 505 may be presented to the user 120. On the user interface 505, an object 510 may be presented. For example, the object 510 may include a word “flower.” Specifically, the object “flower” may be selected to teach the user 120 to create an association between the word “flower” and flowers in real life. Along with presenting the object 510, a first image 515 showing a first type of a flower may be presented to the user 120.

The AR application 575 may have a plurality of images for each object present in a skill development plan. Specifically, the AR application 575 may store a plurality of images of different flowers, such as the first image 515, the second image 520, the third image 525, and the fourth image 530. Each of the first image 515, the second image 520, the third image 525, and the fourth image 530 may explain the object 510.

The images may be stored in a database associated with the AR application 575. Though FIG. 5 shows a gallery 535 of images associated with the object 510 shown on the user interface 505, the gallery 535 may not be shown to the user 120 along with presenting the first image 515. In other words, only the object 510 and one image, such as the first image 515, may be presented to the user 120 on the user interface 505.

After presenting the first image 515 to the user on the user interface 505, a user interface 540 may be provided to the user 120. On the user interface 540, the same object 510 may be presented to the user 120. Along with presenting the object 510, a second image 525 associated with the object 510 and showing a second type of a flower may be presented to the user 120.

At further steps of the method, the third image 525 and the fourth image 530 associated with the object 510 may be sequentially presented to the user 120 along with presenting the object 510.

Therefore, the user 120 may be presented with an array of images which the user may associate with the object 510. Sequential providing of images showing the object 510 in various shapes, sizes, and colors may stimulate the user 120 to develop the skill of generalization (i.e., the skill of making the correlation of objects of different shapes and colors with the concept of the object).

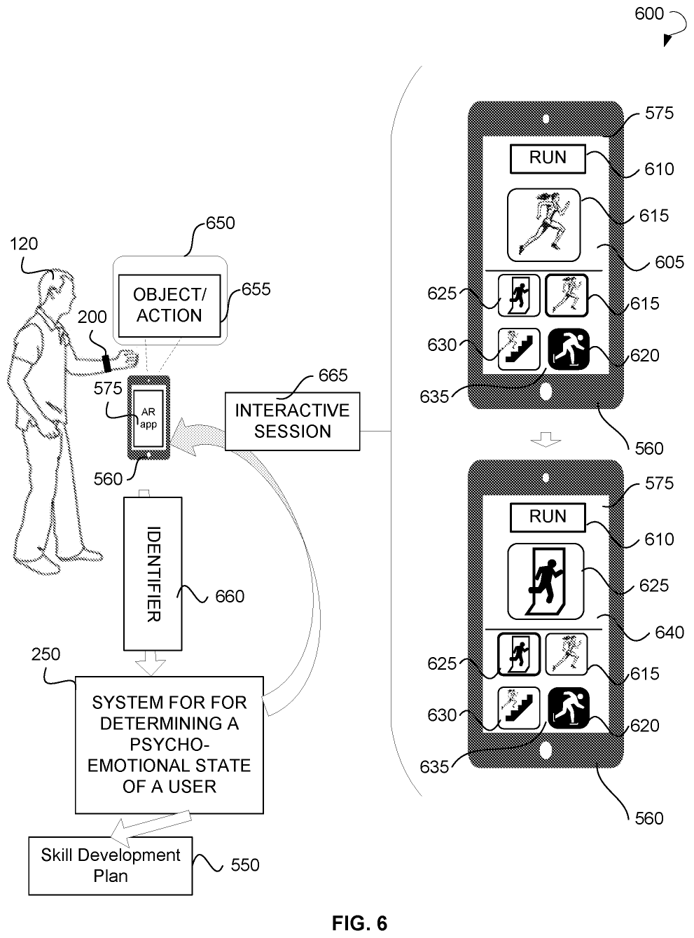

FIG. 6 is a schematic diagram 600 illustrating a method for determining a psycho-emotional state of a user to develop an association between a visual representation of actions and real actions, i.e., the actions per se, according to an example embodiment. In an example embodiment, the method may be used for educating users suffering from autism spectrum disorders and other mental disorders to recognize actions with the help of mobile applications, computer programs, and Internet services. The method solves the problem of inability of users to develop an association between real actions and visual representations of the actions.

The users having aforementioned disorders may be able to create a limited association of actions with visual representations of actions or may be unable to create any association. For example, if an animation shows a running man of a particular age and gender, the user may associate the action “run” only with this specific person of a particular age and gender. The user may be unable to recognize the action of running when the running is performed by any other person or animal. Such peculiarities of perception by the user may limit the ability of the user to understand the real world and use services for training the user, such as via mobile applications, computer programs, and Internet services.

As shown on FIG. 6, a user 120 may wear a wearable device 200. The wearable device 200 may measure physiological parameters of the user 120. The system 250 may analyze the physiological parameters, determine the psycho-emotional state of the user, and select a skill development plan 550 for the user based on the psycho-emotional state.

The skill development plan 550 may include a set of items, such as cards, stickers, or articles. Each of the items may depict an object or an action and may include an AR tag. The AR tag may be an identifier 660 placed on each of the items. The user 120 may activate a camera of the user device 560 and direct the camera view to an item 650 (e.g., a card) depicting an object/action 655. The user device 560 may capture the identifier 660 placed on the item 650 and activate the AR application 575 based on the identifier 660. The AR application 575 may initiate an interactive session 665 associated with the identifier 660 and designed to improve the initial skills with respect to the object/action 655.

The user 120 may interact with an AR application 575 running on a user device 560. A user interface 605 may be presented to the user 120. On the user interface 605, an action 610 may be presented. For example, the action 610 may include a word “run.” Specifically, the action “run” may be selected to teach the user 120 to create an association between the word “run” and the action of running in real life. Along with presenting the action 610, a first image 615 showing a running woman may be presented to the user 120.

The AR application 575 may have a plurality of images for each action present in a skill development plan. Specifically, the AR application 575 may store a plurality of images showing the action of running, such as the first image 615, the second image 620, the third image 625, and the fourth image 630. Each of the first image 615, the second image 620, the third image 625, and the fourth image 630 may explain the action 610.

In an example embodiment, each of the first image 615, the second image 620, the third image 625, and the fourth image 630 may include an animated ‘moving’ image showing the action of running in various scenarios. The images may be stored in a database associated with the AR application 575.

Though FIG. 6 shows a gallery 635 of images associated with the action 610 shown on the user interface 605, the gallery 635 may not be shown to the user 120 along with presenting the first image 615. In other words, only the action 610 and one image, such as the first image 615, may be presented to the user 120 on the user interface 605.

After presenting the first image 615 to the user on the user interface 605, a user interface 640 may be provided to the user 120. On the user interface 640, the same action 610 may be presented to the user 120. Along with presenting the action 610, a second image 625 showing a running man may be presented to the user 120.

At further steps of the method, the third image 625 and the fourth image 630 associated with the action 610 may be sequentially presented to the user 120 along with presenting the action 610.

Therefore, the user 120 may be presented with an array of images which the user may associate with the action 610. Sequential providing of images showing the action 610 in various forms may stimulate the user 120 to develop the skill of generalization (i.e., the skill of making the correlation of actions made by different persons or animals, at different locations and conditions with the concept of the action).

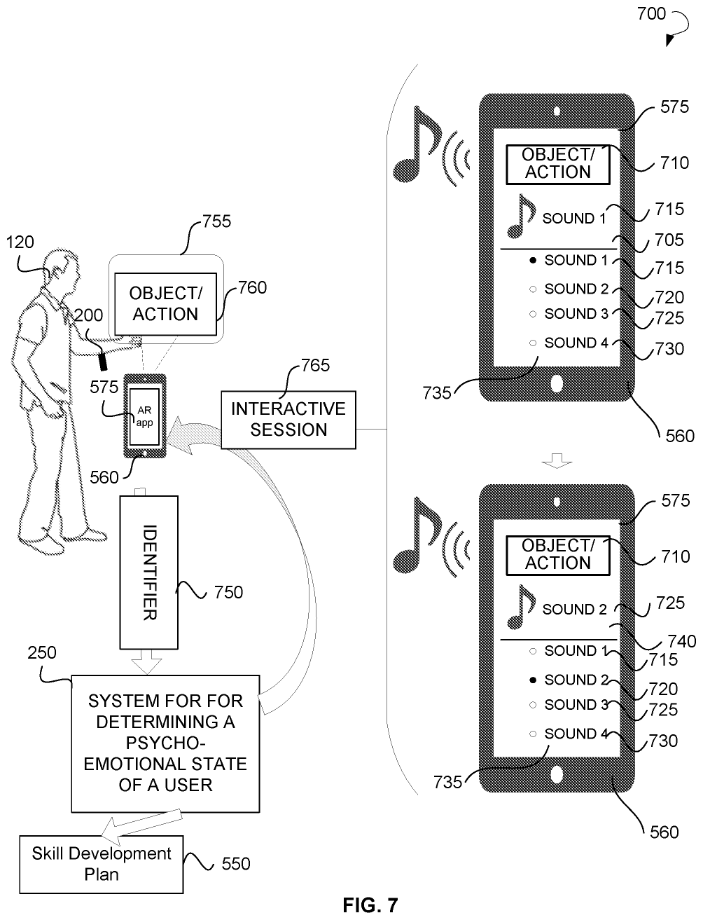

FIG. 7 is a schematic diagram 700 illustrating a method for determining a psycho-emotional state of a user to develop an association between an audible representation (e.g., a spoken word) of objects or actions and real objects or actions or a visual representation of objects or actions, according to an example embodiment. In an example embodiment, the method may be used for educating users suffering from autism spectrum disorders and other mental disorders to recognize objects or actions using mobile applications, computer programs, and Internet services. The method solves the problem of inability of users to develop an association between real objects or actions and audible representation of the objects and actions.

The users having aforementioned disorders may be able to create a limited association of objects or actions with audial representations of objects or actions or may be unable to create any association. For example, if a speaker or a person pronounces a word that means an object or an action, the user may associate the object or the action only with a voice of this particular speaker or person. Specifically, the user may associate the object or the action only with a voice having specific characteristics, such as pitch of a tone and speed of speech.

The user may be unable to recognize the object or action when the object or action is presented by a voice of any other person. Such peculiarities of perception by the user may limit the ability of the user to understand the world and use services for training the users, such as via mobile applications, computer programs, and Internet services.

As shown on FIG. 7, a user 120 may wear a wearable device 200. The wearable device 200 may measure physiological parameters of the user 120. The system 250 may analyze the physiological parameters, determine the psycho-emotional state of the user, and select a skill development plan 550 for the user based on the psycho-emotional state.

The skill development plan 550 may include a set of items, such as cards, stickers, or articles. Each of the items may depict an object or an action and may include an AR tag. The AR tag may be an identifier 750 placed on each of the items. The user 120 may activate a camera of the user device 560 and direct the camera view to an item 755 (e.g., a card) depicting an object/action 760. The user device 560 may capture the identifier 750 present on the item 755 and activate the AR application 575 based on capturing of the identifier 750. The AR application 575 may initiate an interactive session 765 associated with the identifier 750 and designed to improve the initial skills with respect to the object/action 760.

The user 120 may interact with an AR application 575 running on a user device 560. A user interface 705 may be presented to the user 120. On the user interface 705, an object/action 710 may be presented. The object/action 710 may include a word denoting an object or an action. Specifically, the object/action 710 may be selected to teach the user 120 to create an association between the audial representation of the object/action 710 and the object/action in real life. Along with presenting the object/action 710, a first sound 715 may be presented to the user 120. The first sound 715 may be an audio recording of pronunciation of the object/action 710 by a person. The first sound 715 may have a first set of characteristics.

The AR application 575 may have a plurality of audio files for each object/action present in skill development plan 550. Specifically, the AR application 575 may store a plurality of audio files shown as the first sound 715, the second sound 720, the third sound 725, and the fourth sound 730. Each of the first sound 715, the second sound 720, the third sound 725, and the fourth sound 630 may explain the object/action 710.

In an example embodiment, each of the first sound 715, the second sound 720, the third sound 725, and the fourth sound 730 may be an audible representation of the object/action 710, where each of the first sound 715, the second sound 720, the third sound 725, and the fourth sound 730 may have different sets of characteristics, such as different pitch of the sound, different speech speed, and so forth. The audible representations may be stored in a database associated with the AR application 575. Thus, the AR application 575 may use a predetermined algorithm to select different audible representations each time the object/action 710 is presented on the screen. In an example embodiment, the AR application 575 may modify the same audible representation to create different audible representations for each presentation of the object/action 710 on the screen.

Though FIG. 7 shows a gallery 735 of audible representations associated with the object/action 710 shown on the user interface 705, the gallery 735 may not be shown to the user 120 along with presenting the first sound 715. In other words, only the object/action 710 and one sound, such as the first sound 715, may be presented to the user 120 on the user interface 705.

After presenting the first sound 715 to the user on the user interface 705, a user interface 740 may be provided to the user 120. On the user interface 740, the same object/action 710 may be presented to the user 120. Along with presenting the object/action 710, a second sound 720 associated with the object/action 710 and having pitch and speed differing of those of the first sound 715 may be presented to the user 120.

At further steps of the method, the third sound 725 and the fourth sound 730 may be sequentially presented to the user 120 along with presenting the object/action 710.

Therefore, the user 120 may be presented with an array of audible samples which the user may associate with the object/action 710. Sequential providing of audible representations of the object/action 710 using various pitch and speech speed may stimulate the user 120 to develop the skill of generalization (i.e., the skill of making the correlation of objects/actions pronounced by different persons with the concept of the objects/actions).

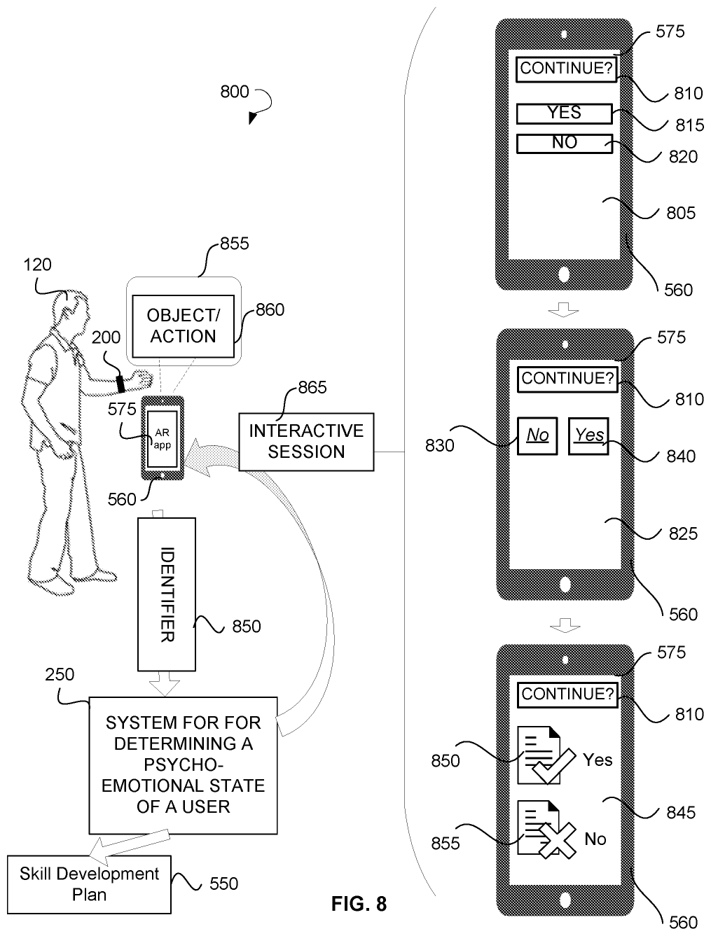

FIG. 8 is a schematic diagram 800 illustrating a method for determining a psycho-emotional state of a user to avoid developing a stereotypical behavior with regard to objects shown on a screen of a computing device, according to an example embodiment. In an example embodiment, the method may be used for educating users suffering from autism spectrum disorders and other mental disorders to avoid a stereotypical behavior when using mobile applications, computer programs, and Internet services. Specifically, the users having mental disorders may have a stereotypical behavior of repetition of the same actions. When the user uses the computing device, windows with buttons that offer a binary choice (Yes/No, Back/Next, Accept/Cancel, etc.) or the choice of one of several options for action (or answer to a question) may be presented to the user. In a case of stereotypical behavior, the user does not think about the meaning of the action being performed, but automatically presses a button located on the same place on the screen as a place of a button the used pressed in response to a previous question. In other words, when the material is repeated, the user mechanically repeats the same movement and gets the correct result. However, in the case providing training and rehabilitation services via computing devices, the user needs to develop not a mechanical skill but an ability to make a conscious choice of the required action.

As shown on FIG. 8, a user 120 may wear a wearable device 200. The wearable device 200 may measure physiological parameters of the user 120. The system 250 may analyze the physiological parameters, determine the psycho-emotional state of the user, and select a skill development plan 550 for the user based on the psycho-emotional state.

The skill development plan 550 may include a set of items, such as cards, stickers, or articles. Each of the items may depict an object or an action and may include an AR tag. The AR tag may be an identifier 850 placed on each of the items. The user 120 may activate a camera of the user device 560 and direct the camera view to an item 855 (e.g., a card) depicting an object/action 860. The user device 560 may capture the identifier 850 present on the item 855 and activate the AR application 575 based on capturing of the identifier 855. The AR application 575 may initiate an interactive session 865 associated with the identifier 850 and designed to improve the initial skills with respect to the object/action 860.

The user 120 may interact with an AR application 575 running on the user device 560. A user interface 805 may be presented to the user 120. On the user interface 805, an object/action 810 may be presented. The object/action 810 may be presented in a form of a sentence, a question (e.g., “Continue?”), and the like. Specifically, the object/action 810 may be selected to teach the user 120 to develop a conscious choice skill. Along with presenting the object/action 810, two options may be provided for selecting by the user 120, namely the first button 815 and the second button 820. The user 120 may respond to the question asked in the object/action 810 by pressing any of first button 815 and the second button 820.

The next time the object/action 810 is repeatedly presented to the user 120, a user interface 825 may be provided to the user 120. On the user interface 825, the same object/action 810 may be presented to the user 120. Along with presenting the object/action 810, two options may be provided for selecting by the user 120, namely the first button 830 and the second button 840. The appearance (e.g., shape, size, color) and/or location on the screen of the first button 830 and the second button 840 may differ from the appearance and location on the screen of the first button 815 and the second button 820.

Similarly, the next time the object/action 810 is repeatedly presented to the user 120, a user interface 845 may be provided to the user 120. On the user interface 845, the same object/action 810 may be presented to the user 120. Along with presenting the object/action 810, two options may be provided for selecting by the user 120, namely the first button 850 and the second button 855. The appearance (e.g., shape, size, color) and/or location on the screen of the first button 850 and the second button 855 may differ from the appearance and location on the screen of the first button 815 and the second button 820, as well as from the appearance and location on the screen of the first button 830 and the second button 840. Thus, the AR application 575 may use a predetermined algorithm to select different types of elements (e.g., rectangular buttons and round buttons), font types, colors, sizes, and different variants of disposition of the elements on the screen of the user device 560 each time the object/action 810 is presented on the screen.

Therefore, each time the user 120 is presented with the object/action 810, the user 120 cannot repeat an unconscious selection of a button the user 120 selected last time but needs to make a conscious choice of the button to correctly answer the question represented by the object/action 810.

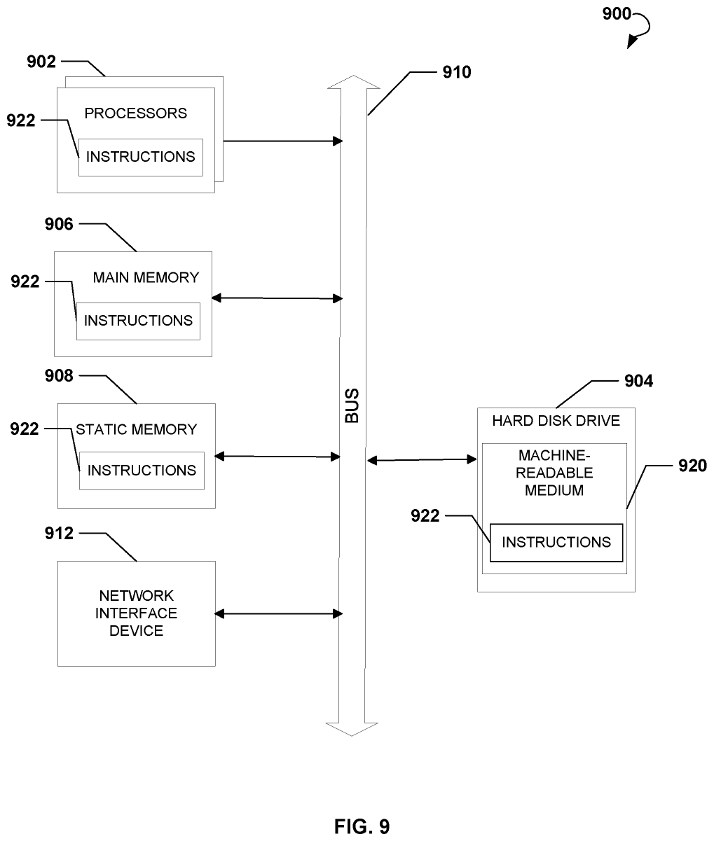

FIG. 9 shows a diagrammatic representation of a computing device for a machine in the exemplary electronic form of a computer system 900, within which a set of instructions for causing the machine to perform any one or more of the methodologies discussed herein can be executed. In various exemplary embodiments, the machine operates as a standalone device or can be connected (e.g., networked) to other machines. In a networked deployment, the machine can operate in the capacity of a server or a client machine in a server-client network environment, or as a peer machine in a peer-to-peer (or distributed) network environment. The machine can be a PC, a tablet PC, a set-top box, a cellular telephone, a digital camera, a portable music player (e.g., a portable hard drive audio device, such as an Moving Picture Experts Group Audio Layer 3 (MP3) player), a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 900 may include a processor or multiple processors 902, a hard disk drive 904, a main memory 906 and a static memory 908, which communicate with each other via a bus 910. The computer system 900 may also include a network interface device 912. The hard disk drive 904 may include a computer-readable medium 920, which stores one or more sets of instructions 922 embodying or utilized by any one or more of the methodologies or functions described herein. The instructions 922 can also reside, completely or at least partially, within the main memory 906 and/or within the processors 902 during execution thereof by the computer system 900. The main memory 906 and the processors 902 also constitute machine-readable media.

While the computer-readable medium 920 is shown in an exemplary embodiment to be a single medium, the term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media. Such media can also include, without limitation, hard disks, floppy disks, NAND or NOR flash memory, digital video disks, Random Access Memory, Read-Only Memory, and the like.

The example embodiments described herein may be implemented in an operating environment comprising software installed on a computer, in hardware, or in a combination of software and hardware.

In some embodiments, the computer system 900 may be implemented as a cloud-based computing environment, such as a virtual machine operating within a computing cloud. In other embodiments, the computer system 900 may itself include a cloud-based computing environment, where the functionalities of the computer system 900 are executed in a distributed fashion. Thus, the computer system 900, when configured as a computing cloud, may include pluralities of computing devices in various forms, as will be described in greater detail below.

In general, a cloud-based computing environment is a resource that typically combines the computational power of a large grouping of processors (such as within web servers) and/or that combines the storage capacity of a large grouping of computer memories or storage devices. Systems that provide cloud-based resources may be utilized exclusively by their owners or such systems may be accessible to outside users who deploy applications within the computing infrastructure to obtain the benefit of large computational or storage resources.

The cloud may be formed, for example, by a network of web servers that comprise a plurality of computing devices, such as the computer system 900, with each server (or at least a plurality thereof) providing processor and/or storage resources. These servers may manage workloads provided by multiple users (e.g., cloud resource customers or other users). Typically, each user places workload demands upon the cloud that vary in real-time, sometimes dramatically. The nature and extent of these variations typically depends on the type of business associated with the user.

Thus, wearable devices, methods, and systems for determining a psycho-emotional state of a user are described. Although embodiments have been described with reference to specific exemplary embodiments, it will be evident that various modifications and changes can be made to these exemplary embodiments without departing from the broader spirit and scope of the present application. Accordingly, the specification and drawings are to be regarded in an illustrative rather than a restrictive sense.

What is claimed is:

1. A wearable device for determining a psycho-emotional state of a user, the wearable device comprising:
a plurality of sensors configured to measure physiological parameters of the user;
an evaluation and testing module configured to evaluate and test the user; and
a processing module configured to:
analyze the physiological parameters of the user during one of the evaluation or the testing; and
based on the analysis, determine the psycho-emotional state of the user;
based on the psycho-emotional state, determine changes in the psycho-emotional state of the user; and
provide recommendations based on the changes in the psycho-emotional state of the user.

2. The wearable device of claim 1, wherein the evaluation and testing module is configured to:
perform an initial evaluation of the user to establish initial skills with respect to an object or an action;
based on results of the evaluation, select a skill development plan for the user designed to improve the initial skills with respect to the object or the action;
provide, to the user, an augmented reality (AR) application for an AR-enabled user device;
provide the object or the action to the user according to the skill development plan;
determine that the user is perceiving the object or the action via the AR-enabled user device, the object or the action being associated with an identifier;
ascertain the identifier associated with the object or the action by the AR application;
based on the identifier, activate, via the AR application, an interactive session designed to improve the initial skills with respect to the object or the action; and
perform a follow-up evaluation of the user to establish an improvement in the at least one of the initial skills.

3. The wearable device of claim 2, wherein the initial evaluation includes identifying a disorder, the disorder including at least one of the following: an autism spectrum disorder, a speech disorder, and a mental disorder.

4. The wearable device of claim 2, wherein the interactive session includes:
presenting a word; and
displaying a plurality of further objects or actions related to the word.

5. The wearable device of claim 2, wherein the interactive session includes repeated pronunciations of a word by varying voices having different voice parameters, the voice parameters including one or more of a speed and a vocal range.

6. The wearable device of claim 2, wherein the interactive session includes presenting buttons having varying parameters, the parameters including a shape, a color, a size, a font, and a location on a screen of the AR-enabled user device.

7. The wearable device of claim 2, wherein the interactive session includes presenting, to the user via the AR-enabled user device, an animation designed to explain a meaning of the object or the action.

8. The wearable device of claim 2, wherein the interaction includes:
providing a test to the user;
receiving a response from the user;
based on the response, selectively providing additional objects, actions, or tests to reinforce the response.

9. The wearable device of claim 2, wherein the object or the action is associated with a card and a sticker.

10. The wearable device of claim 2, wherein the AR-enabled user device includes a smartphone or a tablet.

11. The wearable device of claim 2, wherein the changes are determined based on a comparison of the physiological parameters of the user to historical physiological parameters of the user.

12. The wearable device of claim 11, further comprising a user interface configured to visualize the changes.

13. The wearable device of claim 1, wherein the wearable device is a bracelet.

14. The wearable device of claim 1, wherein the user is diagnosed with a medical condition, the medical condition including one of a chronic disease, a mental condition, and an autism spectrum disorder.

15. The wearable device of claim 1, wherein the psycho-emotional state is indicative of one of a physical exertion, an emotional exertion, a concentration, an emotional state, a sensory overload, an excitement, and an apathy.

16. The wearable device of claim 1, wherein the physiological parameters include one or more of a pulse, a blood pressure, a blood oxygen level, body temperature, and a conductivity of skin.

17. A method for determining a psycho-emotional state of a user during evaluation, the method comprising:
providing a plurality of sensors configured to measure physiological parameters of the user during the evaluation;
evaluating and testing the user;
analyzing the physiological parameters of the user during one of the evaluation or the testing;
based on the analysis, determining the psycho-emotional state of the user;
based on the psycho-emotional state, determining changes in the psycho-emotional state of the user; and
providing recommendations based on the changes in the psycho-emotional state of the user.

18. The method of claim 17, wherein the wearable device is a bracelet.

19. The method of claim 17, wherein the user is diagnosed with a medical condition, the medical condition including one of a chronic disease, a mental condition, and an autism spectrum disorder.

20. A non-transitory computer readable storage medium having embodied thereon a program, the program being executable by a processor to perform a method determining a psycho-emotional state of a user during evaluation, the method comprising:
providing a plurality of sensors configured to measure physiological parameters of the user during the evaluation;
evaluating and testing the user; and
analyzing the physiological parameters of the user during one of the evaluation or the testing; and
based on the analysis, determining the psycho-emotional state of the user;
based on the psycho-emotional state, determining changes in the psycho-emotional state of the user; and
providing recommendations based on the changes in the psycho-emotional state of the user.
